(19) United States
(12) Patent Application Publication
(10) Pub. No.: US 2024/0382721 A1
(43) Pub. Date: Nov. 21, 2024

(54) HYDROCEPHALUS SHUNT

(71) Applicant: FREEFLOW MEDICAL DEVICES LLC, Lancaster, PA (US)

(72) Inventors: Andrew K. Jones, Lancaster, PA (US); Saibal Bandyopadhyay, Lancaster, PA (US)

(21) Appl. No.: 18/688,453

(22) PCT Filed: Sep. 2, 2022
(86) PCT No.: PCT/US2022/042520

§ 371 Date: Mar. 1, 2024

Related U.S. Application Data
(60) Provisional application No. 63/240,585, filed on Sep. 3, 2021.

(51) Int. Cl. A61M 27/00; A61L 29/06

(52) U.S. Cl. CPC A61M 27/006; A61L 29/06; A61M 2207/10

(57) ABSTRACT

The present disclosure provides a hydrocephalus shunt including a ventricular catheter portion having an omniphobic surface. The ventricular catheter portion comprising: a proximal end portion and a distal end portion; wherein the proximal end portion is comprised of fluoropolymer tubing and the distal end portion is comprised of tubing that is comprised of a second material selected from the group consisting of a non-fluorinated polymer, non-perfluorinated polymer, silicone elastomer, fluorosilicone elastomer, or perfluorosilicone elastomer.

This application claims the benefit of U.S. Provisional Patent Application No. 63/240,585, filed Sep. 3, 2021, entitled Hydrocephalus Shunt.

### I. FIELD OF INVENTION

The present invention is related to a hydrocephalus shunt, in particular a hydrocephalus shunt having a ventricular catheter portion configured to resist clogging and blockage due to the adhesion and buildup of tissue, cell, proteins, and related biological materials.

### II. BACKGROUND

Hydrocephalus, excess accumulation of cerebrospinal fluid (CSF) in the brain, is caused by many pathological states like premature birth, traumatic brain injury, stroke, meningitis, and age. Chronically elevated intracranial pressure as a result of hydrocephalus damages brain tissue by, for example, stretching and compressing sensitive brain regions against the skull, leading to long-term neurological deficits in many patients. In infants, hydrocephalus may cause concomitant developmental issues that plague the patient for life. In adults, hydrocephalus may cause dementia, gait imbalance, and incontinence.

Hydrocephalus shunts (ventricular catheters) have been used for the treatment of hydrocephalus to drain fluid from the ventricle into either the peritoneum or another location (body cavity) outside the cranium where the fluid can be absorbed. Typical shunts currently in use are composed of two polydimethylsiloxane (PDMS, silicone) catheters connected by a pressure operated valve (e.g., a one-way valve) that diverts fluid out of the cerebral cavity. The valves may be programmable or non-programmable to regulate the pressure cerebral fluid pressure. Presently, shunts fail in an overwhelming majority of patients in which they are implanted to drain excess cerebrospinal fluid from the cerebral ventricles.

One common cause of shunt failure results from tissue obstruction of the holes in the shunt's ventricular catheter portion that admit CSF into the shunt. Accordingly, there is a clear need to improve the ventricular catheter to prevent tissue obstruction thereby avoiding the cost, pain, and tissue trauma associated with repeated surgeries to replace obstructed shunts. Astrocytes and microglia cells are the dominant cell types that bind directly to hydrocephalus shunts (e.g., the ventricular catheter portion) that results in shunt obstruction. Those cell types are ubiquitous on cerebral shunts following a period of time after implantation, and their number, reactivity, and proliferation increase on failing or failed shunts. In addition, the initially bound cells also create a “glue” or matrix (e.g., of adsorbed proteins) to which additional microglia or other cells and tissues subsequently bind resulting in shunt blockage.

Even when infiltrating ventricular wall cells are present in and/or on a hydrocephalus shunt and involved in its blockage, frequently the blockage is predominately comprised of newly proliferated astrocytes. Accordingly, the initial influx of astrocytes and microglia cells in and around the shunt system is a crucial phase of shunt failure. As indicated above, binding of astrocytes and microglia cells to the shunt is dependent on a layer of adsorbed proteins, without which microglial cell attachment, and the subsequent binding of other cells, does not readily occur. In view of the foregoing, there is a need in the art for improved hydrocephalus shunts that offer greater resistance to blockage which may be achieved by, for example, manipulating the catheter surface chemistry so that the proteins leading to microglia cell attachment are not adsorbed.

### III. SUMMARY OF THE INVENTION

A typical hydrocephalus shunt includes a proximal catheter portion (alternatively referred to as a ventricular catheter portion) that may be inserted into the ventricle of the brain, a valve which controls CSF out flow from the ventricle through the catheter, and a distal catheter portion which drains the CSF fluid from the valve to the peritoneum. The valve may be a one-way valve that prevents back flow of fluid into the brain ventricle(s) and may provide a back pressure consistent with normal physiological CSF pressure.

The present invention includes and provides for ventricular catheters having a slippery omniphobic coating, and an improved hydrocephalus shunt incorporating the ventricular catheters having the slippery omniphobic coating. The omniphobic coating comprises a fluorinated liquid associated with a fluoropolymer surface (e.g., the surface of a fluoropolymer tube or a tube with a fluoropolymer coating). As discussed below the fluorinated liquid may be a perfluorinated liquid, such as a perfluoroalkane. Moreover, the fluoropolymer may be a perfluoropolymer. The slippery omniphobic coating resists attachment of proteins and/or other biological molecules present in CSF by which microglia attach to the catheter, and consequently resists attachment of microglia cells and the concomitant shunt clogging. The omniphobic coating may have a static contact angle with water greater than 110° or 120° measured at 22° C. and may also have a static contact angle with decane and/or hexadecane greater than about 90° at 22° C.

### IV. BRIEF DESCRIPTION OF THE DRAWINGS

FIG. 1 is a schematic depiction of an embodiment of a hydrocephalus shunt of the present invention.

FIG. 2 is a schematic cross-sectional illustration of an embodiment of a ventricular catheter portion 16 of the hydrocephalus shunt of FIG. 1.

FIG. 3 is a cross-sectional illustration of a second embodiment of a ventricular catheter portion 16 of the hydrocephalus shunt of FIG. 1 with overlap 22 formed by tube 20 overlapping tube 24. The tubes may be joined by an adhesive (glue) in the region of overlap and may be densified by the adhesive (e.g., silicone adhesive) infused into the porous polymers present in the region of overlap 22.

FIG. 4 is a cross-sectional illustration of a third embodiment of a ventricular catheter portion 16 of the hydrocephalus shunt of FIG. 1 with overlap 22 formed by insertion of tube 20 into tube 24 (tube 24 overlaps tube 20). The tubes may be joined by an adhesive (glue) in the region of overlap and may be densified by the adhesive (e.g., silicone adhesive) infused into the porous polymers present in the region of overlap 22.

### V. DETAILED DESCRIPTION

### a. Hydrocephalus Shunts and Their Ventricular Catheter Portions

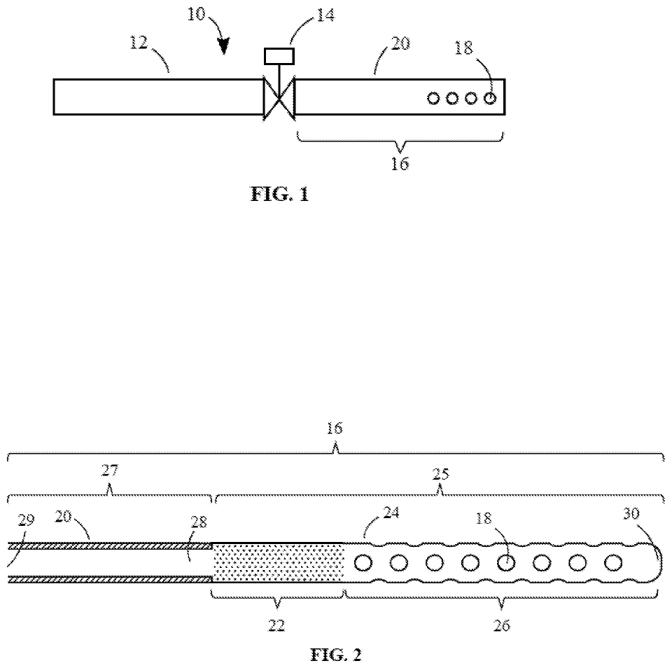

As depicted in FIG. 1, a hydrocephalus shunt 10 of the present invention is comprised of 3 major parts or components: (1) A ventricular catheter portion 16 a portion of which is inserted into the ventricle of the brain, (2) a valve 14 that controls CSF flow from the ventricle through the catheter, and (3) a distal catheter 12 which drains the CSF fluid from the valve (e.g., to the peritoneum or another body cavity). The proximal tip 26 of the ventricular catheter portion (see e.g., FIGS. 2-4) which sits in the ventricle when implanted (e.g., about 2 cm in length), has a series of holes 18 about 0.5 mm to 1 mm in diameter (e.g., in the lateral side of the tubing forming the tip) that place the exterior portion of the proximal tip in fluid communication with the interior of lumen 28 of the shunt and permits cerebrospinal fluid (CSF) to drain from the ventricle by flowing out through the end of lumen 29, which as shown in FIG. 1 is connected to valve 14.

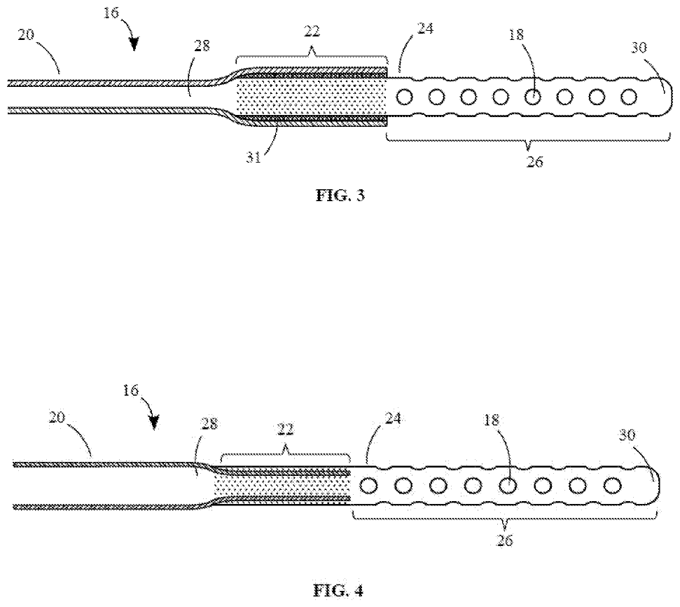

In the embodiment shown in FIG. 2 the ventricular catheter portion 16 is comprised of two sections: a distal end portion 27 comprised of tubing (e.g., silicone tubing) 20 for attachment to valve 14, and a proximal end portion 25. The proximal end portion 25 is comprised of a tube or tubing 24. Tube 24 is comprised of a fluoropolymer (e.g., a fluoropolymer tubing) that has an external surface, at least a portion of which is comprised of the fluoropolymer (e.g., ePTFE). The proximal end portion 25 terminates in a proximal tip 26 of the ventricular catheter portion. The proximal end portion 25 and the distal end portion 27 may be connected by overlapping tube 24 with tubing 20 (e.g., silicone tubing) using the radial forces generated by stretching or compressing tubing 20 relative to tube 24. See FIG. 3 and FIG. 4. The overlap may extend at least about 2 cm, or at least about 3 cm (e.g., about 2.0 to about 3.5 cm, or about 2.5 to about 3.5 cm), although other overlap lengths are discussed below. Silicone or fluorosilicone glue may be used between tube 24 and tubing 20 where they join or overlap 22 to bind them together. The glue (adhesive) may be absorbed completely into pores of the tubing but may also form a thin layer between the tubes (see e.g., 31 in FIG. 3). The junction or overlap may result in the formation of a densified portion of tube 24 infused with silicone when the tube comprises a porous polymer such as ePTFE. Silicone glue may bond with the tubing 20 and/or tube 24. Where the tubing 20 and/or tube 24 is porous and glue becomes infused into the pores, the glue may also become mechanically trapped upon curing. leading to an additional mechanism of attachment. In an embodiment, the distal end portion 27 may be comprised of a flexible silicone elastomer or the like and optionally has a length from about 2 to about 30 cm (e.g., from about 3 to about 15 cm), overlap 22 may comprise ePTFE or another fluoropolymer potentially densified with silicone when porous, and proximal tip 26 may be comprised of a ePTFE or another porous fluoropolymer and optionally has a length from about 2 to about 4 cm (e.g., from about 2.5 to 3.5 cm such as about 3 cm).

In a second embodiment, the ventricular catheter portion 16 comprises tubing 20, which is comprised of a fluoropolymer (e.g., a porous fluoropolymer such as ePTFE). Fluoropolymer tubing 20 of the distal end portion 27 may be joined to the tube 24 of the proximal end portion 25 using, for example, a segment of flexible tubing (e.g., silicone or polyurethane) into which the tubing of the distal and proximal end portions are inserted.

In the embodiment, ventricular catheter portion 16, including tubing 20, proximal end portion 25, and proximal tip 26 is comprised of a single length of fluoropolymer tubing (e.g., porous fluoropolymer tubing such as ePTFE). At least a portion of the tubing's surface may be comprised of a porous fluoropolymer (e.g., ePTFE) that when coated with a fluorinated liquid (e.g., perfluorodecalin) may present a slippery surface to which cells and proteins do not readily adhere. Where the a distal end portion 27 is comprised of a fluoropolymer that is not elastic (e.g., ePTFE), the distal end can be joined to a valve using a segment of flexible tubing (e.g., 2-5 cm in length). The segment of flexible tubing may be slide over the end of tube 20 at one end and a barbed connector end of a valve creating a continuous fluid connection between fluoropolymer tube 20 and the valve. The segment of flexible tubing may be held in place by the forces generated by stretching it over the distal end tube 20 and/or barbs of the valve's barbed end, and/or by an adhesive such as a UV, thermal, or moisture cure silicone.

As noted above, the proximal tip 26 of the ventricular catheter portion 16 is provided with holes 18 (sometimes referred to as drainage holes). In some embodiments the proximal tip 26 has rows of holes (e.g., 4 rows of eight holes) about 0.4 mm to about 1 mm in diameter through which fluid can enter into the ventricular catheter portion of the shunt. See FIGS. 1 to 4. The diameter of the holes 18 may be independently selected, and may be, for example. about 0.4 mm to about 0.6 mm, about 0.6 mm to about 0.75 mm, or about 0.75 mm to about 1 mm. In an embodiment the diameter of each drainage hole 18 varies from about 0.3-0.4 mm, 0.4-0.5 mm, 0.5-0.6 mm, 0.6-0.7 mm, 0.7-0.8 mm, 0.8-0.9 mm, 0.9-1.0 mm, 1.0-1.1 mm, or 1.1-1.2 mm.

The number of holes 18 in proximal tip 26 may vary. For example, the number of holes may be from 4 to 8, from 8 to 12, from 12 to 16, from 16 to 20, from 20 to 24, from 24 to 28, from 28 to 32, from 32 to 36, or from 36 to 40. Drainage holes present in the proximal tip 26 may be arranged in a pattern, such as rows. For example, there may be two, three, or four rows; such as four rows with up to eight drainage holes in each row.

The number, size, shape, and location/orientation of the holes 18 in proximal tip 26 mentioned above are non-limiting, and, for example, other sizes and/or shapes (e.g., slits) may suitably be used. The tip of the fluoropolymer tube (e.g., ePTFE tube) 24 at the end of the catheter 16 may be sealed. The seal 30 may be formed with an ePTFE graft to the fluoropolymer tube 24, which may be sintered in place, closing the end of the shunt 10. The graft may be in the form of a fluoropolymer plug or fluoropolymer cap that is sintered in place. Alternatively, the tip may be sealed by heating and forming the tip or by forming a seal by curing a silicone, fluorosilicone, or perfluorosilicone composition (e.g., a silicone glue).

The inner diameter (ID) of the ventricular catheter portion 16 may be about 1.3 mm (e.g., about 1 mm to about 1.8 mm); however, it does not have to be a single consistent value at all points along the length of the ventricular catheter portion. The outer diameter (OD) of ventricular catheter portion 16 may be about 3 mm (e.g., about 2.2 to about 3.3 mm) with the difference in the ID and OD being consumed by the lateral wall of the tubing (e.g., the wall thickness equals approximately one-half of the difference in the ID and OD). Holes may be arranged with 8 holes per row with a total of 4 rows arranged along the length of the proximal tip (see, e.g., FIG. 2). These dimensions are non-limiting, and other dimensions and configurations may suitably be used.

All of the proximal tip 26, or at least a portion of the external surface and/or internal surface of the lumen of the proximal tip 26 of the shunts of the present disclosure are comprised of a fluoropolymer (e.g., ePTFE) to allow the fluorinated liquid (e.g., perfluorocarbon) to form at the omniphobic surface. The omniphobic coating may be formed on at least the proximal tip 26, but may also be formed on any other portions of the ventricular catheter portion 16 (e.g., the tube 24 comprised of a fluoropolymer), or any other portions of the shunt 10 that comprise a fluoropolymer surface. Where portions of the shunt are prepared from a porous polymer (e.g., porous fluoropolymer or a porous perfluoropolymer such as ePTFE), the fluorinated liquid may infuse into the pores, which may then act as a reservoir of fluorinated liquid to maintain the omniphobic character of the surfaces coated with the fluorinated liquid.

### b. Composition of the Ventricular Catheter Portion

As indicated, the ventricular catheter portion 16 is comprised of two sections: a distal end portion 27, which is comprised of tubing (e.g., silicone tubing) 20 for attachment to valve 14, and a proximal end portion 25. See e.g., FIG. 2, 3, or 4.

The proximal end portion 25, or at least the proximal tip 26 of the ventricular catheter. is comprised of a fluoropolymer (which includes perfluoropolymers). While the entire proximal end may comprise, consist of, or consist essentially of, a fluoropolymer, at a minimum the exterior surface of the proximal tip 26 has a fluoropolymer coating. The proximal tip 26 may also have a coating of fluoropolymer on its exterior and/or interior.

A variety of fluoropolymers (e.g., perfluoropolymers) are suitable for use in constructing the proximal end portion 25 and its proximal tip 26, the distal end portion 27 including its tubing 20, or any other portions of hydrocephalus shunt 10 (e.g., portions intended to have an omniphobic surface). Non-limiting examples of suitable fluoropolymers include perfluoroalkoxy alkane (PFA), polytetrafluoroethylene (PTFE), fluorinated ethylene propylene (FEP), expanded polytetrafluoroethylene (ePTFE), ethylene fluorinated ethylene propylene (EFEP), ethylene tetrafluoroethylene (ETFE), polyvinylidene fluoride (PVDF), vinylidene fluoride-hexafluoropropylene fluoroelastomer (VF2/HFP), vinylidene fluoride-hexafluoropropylene/tetrafluoro ethylene/hexafluoropropylene fluoroelastomer (VF2/tetrafluoro ethylene/HFP terpolymer), fluoroelastomers, perfluoroelastomers, and combinations thereof. Such fluoropolymers include porous fluoropolymers and perfluoropolymers (e.g., ePTFE).

Where the ventricular catheter portion 16 is made out of fluoropolymer (e.g., ePTFE), the length of the ventricular catheter portion may be about 1-2 cm, 2-3 cm, 3-4 cm, 4-5 cm, 5-6 cm, 6-7 cm, 7-8 cm. 8-9 cm, 9-10 cm, 10-11 cm, 11-12 cm, 12-13 cm, or 13-14 cm.

Flexibility of the shunt and case of attaching the ventricular catheter portion 16 to valve 14 are desirable properties that facilitate implantation in a subject. Those properties permit the use of a variety of materials in the construction of the distal end portion 27, which may be comprised of the same fluoropolymer material as the proximal end portion. Alternatively, the distal end portion 27 may be comprised of a flexible and elastic material including non-fluorinated polymer (e.g., non-fluorinated polyurethanes), non-perfluorinated polymer, a silicone elastomer, a fluorosilicone elastomer or a perfluorosilicone elastomer. Tubing 20, when prepared from elastic materials, not only provides flexibility, but also permits facile attachment of ventricular catheter portion 16 to the hydrocephalus shunt valve 14. The tubing seals to the valve and can produce a fluid tight connection resulting from the radial force generated by stretching or compressing the tubing 20 relative to the stem of the valve. Where the valve stem over which tubing 20 is placed has barbs, flexible tubing, such as tubing comprising a silicone elastomer, can conform to the barbed surface providing a secure mechanical attachment. Moreover, a surgical knot can be tied over tubing 20 when it is a flexible/compressible material to further ensure it remains attached to the valve.

### c. Joining of the Ventricular Catheter Portions

The proximal end portion 25 and the distal end portion 27 are joined to each other so that their lumens are in continuous fluid communication from the proximal tip 26 to and through the distal portion to the open end of the lumen 29. The proximal and distal end portions may be joined (secured to each other) end-to-end, or by overlapping one of the ends over the other to create a region 22 where they are joined or overlap. Joining of the proximal and distal end portions may be accomplished mechanically and/or through bonding.

Mechanical joining of the proximal and distal end portions (25 and 27) may be accomplished by overlapping the tubing 20 of the distal end portion 27 over tube 24 or vice versa. The radial forces generated by stretching or compressing the tubing 20 relative to tube 24 in the region of overlap serve to mechanically bind the proximal and distal end portions. For example, where tubing 20 is made of an elastic material (e.g., silicone elastomer, fluorosilicone elastomer or perfluorosilicone elastomer), tube 24 may be joined to it by using tubing suitably sized (the OD of tube 24 is suitably larger than the ID of tubing 20), such that when tube 24 is pressed inside the lumen of tubing 20 (compression fitted) it is forced to stretch, generating a compressive force on tube 24, joining the tubes and forming a fluid tight seal. See e.g., FIG. 3. The specific differences in diameter of tubing 20 and tube 24 required to join the proximal and distal end portions will depend on several factors including the elasticity and rigidity of the materials, and how long a section of overlap is desired or required to form a mechanically stable joint that is fluid tight.

Where tubing 20 is comprised of an elastic material (e.g., silicone elastomer) that is to be externally fitted on (overlap) tube 24, (see, e.g., FIG. 3) the inner diameter of tubing 20 may be less than the outer diameter of tube 24 by 0% to 1%, 1% to 5%, 5% to 10%, or 10 to 20% over the length where tube 24 is to be inserted. This may create an insertion pocket at the end of tubing 20, with the remaining inner diameter of the silicone tube matched with the inner diameter of tubing 24. Tubing with an insertion pocket may be produced by extrusion molding followed by overmolding the larger diameter section to create the pocket.

Where the tube 24 comprises a porous fluoropolymer and is externally fitted onto (overlaps) tubing 20 (see e.g., FIG. 4), which is comprised of an elastic material (e.g., a silicone elastomer), the outer diameter of tubing 20 may be matched with the inner diameter of tubing 24. The adhesion between the two tubes may be reinforced by bonding with, for example, a silicone glue.

When joining the proximal and distal end portions by overlapping tubing, a segment of tubing that overlaps with both tubing 20 and 24 may be interposed at the location of the joint or overlap 22. In such a case the segment of tubing, for example, may be compression fitted into the lumen of both tubing 20 and 24, or alternatively, tubing 20 and 24 may be compression fitted into the lumen of the interposed segment of tubing. The interposed segment of tubing may be prepared from any material suitable for the preparation of tubing 20 or 24.

As an alternative to, or in addition to, mechanical joining, the proximal and distal end portions (25 and 27) may be joined by bonding the tubing 20 and 24 together either end-to-end without an overlap, or by overlapping. Regardless of the geometry of the joint, bonding of tubing 20 and 24 may be accomplished through the use of adhesives (e.g., glues that are moisture or heat cured) and/or by heating. In such a case the resulting ventricular catheter portion 16 may appear as in FIG. 2, however, the densification of section 22 will dependent on the amount of silicone adhesive or glue that manages to infuse into the porous fluoropolymer tubing 24. As discussed below, overmolding may also be used to prepare ventricular catheter portion 16 as appears in FIG. 2.

Heat may be applied e.g., at the location of the joint or overlap 22 to fuse the material(s) of tubing 20 and tubing 24. Heat may be provided from any suitable source, such as a stream of hot air, a hot metal element brought close to or in contact with the tubing 20 and 24, or a light source (e.g., laser light). Where necessary, a support may be inserted in the lumen to ensure it maintains its shape and/or size.

Glues (adhesives) such as a silicone or fluorosilicone (e.g., perfluorosilicone) paste or glue, or any other adhesive material which can infuse into the pores of a porous fluoropolymer such as ePTFE, may be used to bond sections of the shunt together (e.g., tubing 20 and 24 and/or or tubing 20 to valve 14) together whether they meet end-to-end without an overlap, or they meet with an overlap. Silicone and fluorosilicone glues suitable for use include heat cured (e.g., using peroxide or Pt catalyst), UV cured, or moisture cured silicone compositions. The glues, which are applied uncured (e.g., as a liquid or paste), may bond to one or both of tubing 20 and/or 24. Where either or both of tubing 20 or 24 is comprised of a porous material (e.g., a porous fluoropolymer such as ePTFE), the uncured glue may also enter into the pores (become infused in the porous material) and upon curing is lodged in the pores leading to an addition mechanism of bonding. Silicone and/or fluorosilicone glues, such as moisture cure silicone glues, may be cured without heating using ambient conditions (e.g., less than 30° C.). Depending on the catalyst system used, other silicone glues may be cured without heating or by heating (e.g., at greater than 30° C. or greater than 40° C.).

In an embodiment, where tubing 24 is made of a porous fluoropolymer (e.g., ePTFE) or comprises a porous fluoropolymer surface, at least the portion of the tubing that is to overlap with tubing 20 (e.g., silicone or fluorosilicone elastomer tubing) is either completely or partially infused with uncured silicone glue (e.g., a fluorosilicone glue or perfluorosilicone glue). Tubing (e.g., silicone tubing) 20 and the section of porous fluoropolymer tubing infused with the uncured silicone are fitted together (compression fitted together) so that the uncured silicone glue contacts both tubing 20 and tubing 24 (i.e., there is uncured silicone glue in contact with both tubes). See FIGS. 3 and 4. In one embodiment describe by FIG. 2 tubing 20 and 24 are joined end-to-end with silicone glue. Where silicone glue is employed to join tubing 20 and 24 the entire end (interior and/or exterior) of porous fluoropolymer tubing 24 may be infused with uncured silicone glue resulting in densification of the tubing during curing). The silicone glue is subsequently cured bonding tubing 20 and 24 together and forming the ventricular catheter portion with a densified section of tubing 22.

An alternatively method of preparing a ventricular catheter portion 16 as shown in FIG. 2 is to employ overmolding. In such a method, at least a portion of the porous fluoropolymer tubing 24 of proximal end portion 25 is infused with uncured silicone elastomer composition (a heat curable liquid silicone composition that cures to form a silicone rubber). The infused proximal portion is then placed in a mold with a mandrel inserted into the lumen of infused tubing 24. The mold is filled with uncured silicone (e.g., of the same or a similar composition as that infused into at least a portion of tubing 24), and the uncured silicone composition exposed conditions under which it is cured (e.g., by heating) to prepare tubing 20 around the mandrel and bonded to tubing 24. Although it is not shown in FIG. 2, a thin layer of cured silicone elastomer (rubber) continuous with tube 20 may be located on the interior and/or exterior of tubing 24 depending on how tightly the mold and mandrel fits against the portion of the porous fluoropolymer tube 24 infused with the uncured silicone prior to curing.

FIGS. 3 and 4 depict other aspects of bonded proximal and distal end portions. In FIG. 3 the external portion of tubing 24 and/or the internal portion of tubing 20 may be infused with uncured silicone glue over all or part of the portion they are to overlap. Tubing 24 is compression fitted into tubing 20 and the silicone glue cured, bonding tubing 20 and 24 together and forming the ventricular catheter portion. In FIG. 4 the internal portion of tubing 24 and/or the external portion of tubing 20 are infused with uncured silicone glue over all or part of the portion they are to overlap. Tubing 20 is compression fitted into tubing 24 and the silicone glue cured, bonding tubing 20 and 24 together and forming the ventricular catheter portion.

Where tubing 24 is comprised of a porous fluoropolymer (e.g., ePTFE) and joined to tubing 20 (e.g., a silicone or fluorosilicone elastomer) by overlapping and bonding, the length of overlap with tubing 20 where silicone glue is infused into tubing 20 and/or 24 may be from 1 to 5 mm, from 5 to 10 mm, from 10 to 20 mm, from 20 to 30 mm, from 30 to 40 mm or from 40 to 50 mm.

Where tubing 24 is comprised of a porous fluoropolymer (e.g., ePTFE) and is bonded to tubing 20 (e.g., silicone or fluorosilicone elastomer tubing), the length of the fluoropolymer tubing bonded to tubing 20 may be from 1 to 5 mm, from 5 to 10 mm, from 10 to 20 mm, from 20 to 30 mm, from 30 to 40 mm or from 40 to 50 mm.

As an alternative to joining sections of the shunt using liquid or paste adhesives (glues) applied to or impregnated into sections of the shunt, porous tape (such as ePTFE tape) may be impregnated with adhesive (glue) may be employed. Some suitable adhesives that may be impregnated (infused) into porous tapes include silicone or fluorosilicone (e.g., perfluorosilicone) adhesives in the form of a liquid or paste. Once the porous tape is impregnated with the adhesive it can be placed or wrapped around the adjoining sections of a shunt to form (e.g., where the sections are joined end-to end or reinforce a joint between the sections. The impregnated tape may also be placed or wrapped around the shunt section to be inserted into an adjoining section, thereby providing a layer of adhesive between the sections when assembled. The adhesive in the tape is then cured to join and/or reinforce the junction between the sections. Silicone and/or fluorosilicone glues, such as moisture cure silicone glues, may be cured while infused into the ePTFE tape without heating using ambient conditions (e.g., less than 30° C.). Depending on the catalyst system used, silicone glues may be cured without applying heat (ambient temperature curing) or by heating (e.g., heating to a temperature greater than 30° C. or greater than 40° C.). UV curable adhesives (e.g., UV-cure silicones) offer a means to rapidly join shunt sections and may be employed where the materials to be joined permit UV light to reach the uncured adhesive (glue).

### d. Fluorinated Liquids and the Formation of Omniphobic Surfaces

In order to create an omniphobic surface on a surface of the shunt (e.g., on the ventricular catheter portion), a liquid comprising one or more fluorinated liquids, one or more perfluorinated liquid, or a combination of one or more fluorinated and/or one or more perfluorinated liquids needs to be applied to (coated on) the surfaces of the shunt where the omniphobic surface is to be formed. The locations where liquid is to be applied should be comprised of a porous fluoropolymer and/or perfluoropolymer (e.g., ePTFE), the pores of which may act as a reservoir for the fluorinated liquid extending the life of the omniphobic surface.

Non-limiting examples of fluorinated liquids that may be used to form the omniphobic surface include liquid fluorocarbons, liquid perfluorocarbons (e.g., perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexa)fluoropropylene oxide), and combinations thereof.

### e. Certain Embodiments

The following embodiments may be used in any combination.
1. A ventricular catheter portion 16 of a hydrocephalus shunt comprising a proximal end portion and a distal end portion:
   wherein the proximal end portion 25 comprising fluoropolymer tubing 24 that terminates at a proximal tip 26 is comprised of a first material selected from the group consisting of a fluoropolymer, a perfluoropolymer, and combinations thereof;
   wherein the distal end portion 27 which is comprised of tubing 20 that is comprised of the first material or a second material that is comprised of a non-fluorinated polymer, non-perfluorinated polymer, silicone elastomer, fluorosilicone elastomer or perfluorosilicone elastomer; and
   wherein the fluoropolymer tubing of the proximal end portion and the tubing of the distal end portion have a lumen 28 with an open end of the lumen 29 distal to the proximal tip 26.
2. The ventricular catheter portion of embodiment 1, wherein the first material comprises one or more of: PFA, PTFE, FEP, ePTFE. EFEP, ETFE, PVDF, VF2/HFP, VF2/tetrafluoro ethylene/HFP terpolymer, a fluoroelastomer, and/or a perfluoroelastomer.
3. The ventricular catheter portion of any preceding embodiment, wherein the first material is a porous fluoropolymer, a porous perfluoropolymer, or a porous fluoropolymer-perfluoropolymer combination.
4. The ventricular catheter portion of any preceding embodiment, wherein the first material comprises ePTFE.
5. The ventricular catheter portion of embodiment 1 or 2, wherein the second material of the catheter comprises an elastomer, silicone (silicone elastomer), fluorosilicone elastomer, perfluorosilicone elastomer, or polyurethane.
6. The ventricular catheter portion of any preceding embodiment, wherein the section where the fluoropolymer tubing 24 and tubing 20 of the distal end portion 27 join or overlap (section 22) extends from 0 to 1 cm, from 1 cm to 2 cm, from 2 cm to 3 cm, from 3 cm to 4 cm, from 4 cm to 5 cm, from 5 cm to 6 cm, from 6 cm to 7 cm, from 7 cm to 8 cm, from 8 cm to 9 cm, from 9 cm to 10 cm, from 10 cm to 11 cm, from 11 cm to 12 cm, from 12 cm to 13 cm, or from 13 cm to 14 cm.
7. The ventricular catheter portion of any preceding embodiment, wherein the tubing 20 of the distal end portion (e.g., tubing 20) is comprised of silicone, fluorosilicone, or perfluorosilicone and is configured to be attached to a valve; and
   wherein the proximal tip 26 (configured to be inserted into cerebral ventricles) is comprised of ePTFE.
8. The ventricular catheter portion of any preceding embodiment, wherein the distal end portion 27 comprises tubing 20 which is comprised of silicone (silicone elastomer), fluorosilicone elastomer, and/or perfluorosilicone elastomer, and the fluoropolymer tubing 24 of the proximal end portion 25 is comprised of ePTFE; and
   wherein the tubing 20 of the distal end portion is mechanically attached to the fluoropolymer tubing 24 to create a ventricular catheter portion having a continuous lumen 28.
9. The ventricular catheter portion of any preceding embodiment, wherein the distal end portion 27 comprises tubing 20 that is comprised of a non-fluorinated silicone.
10. The ventricular catheter portion of any preceding embodiment, wherein the fluoropolymer tubing 24 is comprised of a porous fluoropolymer (e.g., ePTFE) and overlaps tubing 20 of the distal end portion that is comprised of a silicone (silicone elastomer), fluorosilicone elastomer, and/or perfluorosilicone elastomer; and
   wherein a layer of silicone or fluorosilicone paste or glue is located between the fluoropolymer tubing 24 and the tubing 20 of the distal end portion over all or part of the portion that they overlap.
11. The ventricular catheter portion of any preceding embodiment, wherein the fluoropolymer tubing 24 is comprised of a porous fluoropolymer (e.g., ePTFE) and overlaps tubing 20 of the distal end portion that is comprised of a silicone (silicone elastomer), fluorosilicone elastomer, and/or perfluorosilicone elastomer; and
   wherein silicone or fluorosilicone paste or glue is infused into the pores of the fluoropolymer tubing 24 over all or part of the portion that overlaps with the tubing 20 of the distal end portion.
12. The ventricular catheter portion of any of embodiments 1 to 10, wherein the tubing 20 of the distal end portion that is comprised of a silicone (silicone elastomer), fluorosilicone elastomer, and/or perfluorosilicone elastomer overlaps the fluoropolymer tubing 24 that is comprised of a porous fluoropolymer (e.g., ePTFE); and optionally
   wherein a layer of silicone or fluorosilicone paste or glue is located between the fluoropolymer tubing 24 and the tubing 20 of the distal end portion (e.g., over all or part of the portion that they overlap).
13. The ventricular catheter portion of any of embodiments 1 to 10, wherein the tubing 20 of the distal end portion that is comprised of a silicone (silicone elastomer), fluorosilicone elastomer, and/or perfluorosilicone elastomer overlaps the fluoropolymer tubing 24 that is comprised of a porous fluoropolymer (e.g., ePTFE); and optionally
   wherein silicone or fluorosilicone paste or glue is infused into the pores of the fluoropolymer tubing 24 over all or part of the portion that overlaps with the tubing 20 of the distal end portion (e.g., over all or part of the portion that they overlap).
14. The ventricular catheter portion of any preceding embodiment, wherein either the tubing 20 of the distal end portion 27 or the fluoropolymer tubing 24 of the proximal end portion 25 overlaps the other tubing, and the tubing 20 and fluoropolymer tubing 24 are joined together by the mechanical compressive force of the overlapping tubing on the other tubing.
15. The ventricular catheter portion of any of embodiments 10 through 14, wherein the tubing 20 of the distal end portion 27 and the fluoropolymer tubing 24 of the proximal end portion 25 are bonded over all or part of the overlap (e.g., by fusing the tubing materials, curing the silicone or fluorosilicone glue, or fusing the silicone or fluorosilicone paste to tubing 20 and 24, such as by heating).
16. A ventricular catheter portion of any of embodiments 1-4 and 6, wherein the proximal end portion and the distal end portion are comprised of ePTFE having pores.
17. The ventricular catheter portion of embodiment 16, wherein a non-fluorinated polymer is infused into the pores of the ePTFE on at least a portion of the interior of lumen 28 at the open end of the lumen 29 (e.g., to provide a sealing surface for mating to valve 14 through densification of the ePTFE and/or increased friction relative to the ePTFE).
18. The ventricular catheter portion of embodiment 16, wherein a non-fluorinated liner is inserted into at least a portion of the interior of lumen 28 at the open end of the lumen 29 (e.g., contacting and conforming to the portion of the interior wall of tubing 20 and providing a sealing surface for mating to valve 14 through densification of the ePTFE and/or increased friction relative to the ePTFE).
19. The ventricular catheter portion of embodiment 18, wherein the liner is comprised of polyurethane or silicone.
20. The ventricular catheter portion of any preceding embodiment, wherein at least a portion of a surface of the proximal end portion, or a portion of the surface of both the proximal end portion and the distal end portion is coated with one or more fluorinated liquids, one or more perfluorinated liquids, or a combination of one or more fluorinated liquids and/or one or more perfluorinated liquids.
21. The ventricular catheter portion of embodiment 20, wherein the one or more fluorinated liquids, the one or more perfluorinated liquids, or the combination of one or more fluorinated liquids and/or perfluorinated liquids comprises a fluoroalkane or perfluoroalkane (e.g., perfluorodecalin).
22. The ventricular catheter portion of any of embodiments 20 to 21, wherein greater than 50%, 75%, 80%, or 90%, or 100%, of an exterior surface of the proximal end portion, the distal end portion, or both the proximal end portion and the distal end portion is comprised of a fluoropolymer and/or perfluorinated polymer and is coated with the fluorinated liquid. perfluorinated liquid, or a combination of one or more fluorinated liquids and/or one or more perfluorinated liquids.
23. The ventricular catheter portion of any of embodiments 20 to 22, wherein the one or more fluorinated liquids, the one or more perfluorinated liquids, or the combination of one or more fluorinated liquids and/or perfluorinated liquids comprises, consists essentially of, or consists of one or more perfluorinated liquids.
24. The ventricular catheter portion of any of embodiments 20 to 23, wherein the one or more fluorinated liquids, the one or more perfluorinated liquids, or the combination of one or more fluorinated liquids and/or one or more perfluorinated liquids comprises greater than:
   (i) 50%, 60%, 70%, 80%, 90%, 95%, or 98% of one or two fluorinated and/or perfluorinated compounds on a weight basis;
   (ii) 50%, 60%, 70%, 80%, 90%, 95%, or 98% of fluorinated compounds on a weight basis;
   (iii) 50%, 60%, 70%, 80%, 90%, 95%, or 98% of perfluorinated compounds on a weight basis; or
   (iv) 50%, 60%, 70%, 80%, 90%, 95%, or 98% of fluorinated and/or perfluorinated compounds on a weight basis.
25. The ventricular catheter portion of any of embodiments 20 to 24, wherein the one or more fluorinated liquids, the one or more perfluorinated liquids, or the combination of one or more fluorinated liquids and/or one or more perfluorinated liquids comprises: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexa)fluoropropylene oxide, or combinations thereof.
26. A method of making a medical device according to any preceding embodiment, the method comprising preparing all or part of the proximal end portion 25 or the distal end portion 27 by one or more of: machining away excess material, drilling, laser cutting, laser drilling, extrusion, co-extrusion, injection molding, compression molding, melt spinning, electrospinning, dip coating, blowing, foaming, over-molding, and chemical vapor deposition. For example, holes 18 (drainage holes) may be laser cut, drilled, or punched out.
27. The method of embodiment 26, wherein the proximal and/or distal end portion comprise a fluoropolymer and/or perfluoropolymer present on at least a portion of their exterior surfaces, wherein the fluoropolymer or perfluoropolymer containing exterior surface is formed by a method selected from the group consisting of: extrusion, co-extrusion, injection molding, compression molding, melt spinning, electrospinning, dip coating, blowing, foaming, and/or chemical vapor deposition.
28. A ventricular catheter portion of a hydrocephalus shunt made by the method of embodiment 26 or 27.
29. The ventricular catheter portion of a hydrocephalus shunt of embodiments 1 to 25 or 28 packaged in a sterile nonporous container coated with one or more fluorinated liquids, one or more perfluorinated liquids, or a combination of one or more fluorinated liquids and/or one or more perfluorinated liquids.
30. The ventricular catheter portion of a hydrocephalus shunt of embodiments 1 to 19 or 28, packaged in a sterile nonporous container in the absence of one or more fluorinated liquids, one or more perfluorinated liquids, or a combination of one or more fluorinated liquids and/or one or more perfluorinated liquids.
31. The ventricular catheter portion of a hydrocephalus shunt of embodiments 29 or 30, where the package either further comprises or is accompanied by a sterile aliquot of the one or more fluorinated liquids, one or more perfluorinated liquids, or the combination of one or more fluorinated liquids and/or one or more perfluorinated liquids.
32. A hydrocephalus shunt comprising a ventricular catheter portion of any of embodiments 1-25 or 28.
33. The hydrocephalus shunt of embodiment 32, wherein the ventricular catheter portion is attached to a valve 14 (e.g., a surgically implantable valve) at the open end of the lumen 29 permitting the lumen of the ventricular catheter portion 28 to be in fluid communication with a distal catheter 12 (via the valve, see, e.g., FIG. 1).
34. The hydrocephalus shunt of embodiment 33, wherein a portion of the valve providing fluid communication with the lumen 28 of tubing 20 is a hollow tube inserted into the open end of the lumen 29.
35. The hydrocephalus shunt of embodiment 33, wherein the portion of the valve inserted into the open end of the lumen 29 has a barbed exterior surface and the compressive mechanical force of the tubing 20 of the distal end portion on the barbed exterior surface acts to secure and provides a fluid tight connection between the valve and the tubing 20.
36. A method of treatment comprising implanting the hydrocephalus shunt of any of embodiments 32 to 35 in an animal (e.g., in a mammal such as a human).
37. The method of embodiment 36, wherein the ventricular catheter portion 16 of the shunt is implanted in the ventricle of a mammalian brain and ventricle pressure is reduced due to fluid flow out of the ventricle through the catheter.

We claim:

1. A ventricular catheter portion of a hydrocephalus shunt comprising: a proximal end portion and a distal end portion;
wherein the proximal end portion, which is comprised of fluoropolymer tubing terminating in a proximal tip, is comprised of a first material selected from the group consisting of a fluoropolymer, a perfluoropolymer, and combinations thereof;
wherein the distal end portion is comprised of tubing that is comprised of a second material selected from the group consisting of a non-fluorinated polymer, non-perfluorinated polymer, silicone elastomer, fluorosilicone elastomer, or perfluorosilicone elastomer; and
wherein the fluoropolymer tubing of the proximal end portion and the tubing of the distal end portion have a lumen with an open end of the lumen distal to the proximal tip.

2. The ventricular catheter portion of claim 1, wherein the first material comprises one or more of: PFA, PTFE, FEP, ePTFE, EFEP, ETFE, PVDF, VF2/HFP, VF2/tetrafluoro ethylene/HFP terpolymer, a fluoroelastomer, and/or a perfluoroelastomer.

3. The ventricular catheter portion of claim 1, wherein the first material is a porous fluoropolymer, a porous perfluoropolymer, or a porous fluoropolymer-perfluoropolymer combination.

4. The ventricular catheter portion of claim 1, wherein the first material comprises ePTFE.

5. The ventricular catheter portion of claim 3, wherein the second material of the catheter comprises an elastomer, silicone, fluorosilicone, perfluorosilicone, or polyurethane

6. The ventricular catheter portion of claim 5, wherein the section where the fluoropolymer tubing and the tubing of the distal end portion join or overlap extends from 0 to 1 cm, from 1 cm to 2 cm, from 2 cm to 3 cm, from 3 cm to 4 cm, from 4 cm to 5 cm, from 5 cm to 6 cm, from 6 cm to 7 cm, from 7 cm to 8 cm, from 8 cm to 9 cm, from 9 cm to 10 cm, from 10 cm to 11 cm, from 11 cm to 12 cm, from 12 cm to 13 cm, or from 13 cm to 14 cm.

7. The ventricular catheter portion of claim 1, wherein the tubing of the distal end portion is comprised of silicone, fluorosilicone, or perfluorosilicone and is configured to be attached to a valve; and
wherein the proximal tip is comprised of ePTFE.

8. The ventricular catheter portion of claim 1, wherein the distal end portion comprises a tube comprised of a silicone elastomer, fluorosilicone elastomer, and/or perfluorosilicone elastomer, and the fluoropolymer tubing of the proximal end portion is comprised of ePTFE; and
wherein the tubing of the distal end portion is mechanically attached to the fluoropolymer tubing to create a ventricular catheter portion having a continuous lumen.

9. The ventricular catheter portion of claim 8, wherein the distal end portion comprises tubing that is comprised of a non-fluorinated silicone.

10. The ventricular catheter portion of claim 1, wherein the fluoropolymer tubing is comprised of ePTFE and overlaps the tubing of the distal end portion that is comprised of a silicone, fluorosilicone elastomer, and/or perfluorosilicone elastomer; and
wherein a layer of silicone or fluorosilicone paste or glue is located between the fluoropolymer tubing and the tubing of the distal end portion over all or part of the portion that they overlap.

11. The ventricular catheter portion of any preceding claim, wherein the fluoropolymer tubing is comprised of ePTFE and overlaps the tubing of the distal end portion that is comprised of a silicone elastomer, fluorosilicone elastomer, and/or perfluorosilicone elastomer; and
wherein silicone or fluorosilicone paste or glue is infused into the pores of the fluoropolymer tubing over all or part of the portion that overlaps with the tubing of the distal end portion.

12. The ventricular catheter portion of any of claims 1 to 10, wherein the tubing of the distal end portion is comprised of a silicone, fluorosilicone elastomer, and/or perfluorosilicone elastomer and overlaps the fluoropolymer tubing, which is comprised of ePTFE; and optionally
wherein a layer of silicone or fluorosilicone paste or glue is located between the fluoropolymer tubing and the tubing of the distal end portion over all or part of the portion that they overlap.

13. The ventricular catheter portion of claim 12, wherein the tubing of the distal end portion is comprised of a silicone, fluorosilicone elastomer, and/or perfluorosilicone elastomer and overlaps the fluoropolymer tubing, which is comprised of ePTFE; and optionally
wherein silicone or fluorosilicone paste or glue is infused into the pores of the fluoropolymer tubing over all or part of the portion that overlaps with the tubing of the distal end portion.

14. The ventricular catheter portion of any of claims 1 to 10, wherein either the tubing of the distal end portion or the fluoropolymer tubing of the proximal end portion overlaps the other tubing, and they are joined together by a mechanical compressive force of the overlapping tubing on the other tubing.

15. The ventricular catheter portion of claim 14, wherein the tubing of the distal end portion and the fluoropolymer tubing of the proximal end portion are bonded over all or part of the overlap.

16. A ventricular catheter portion of any of claims 1 to 4, wherein the proximal end portion and the distal end portion are comprised of ePTFE having pores.

17. The ventricular catheter portion of claim 16, wherein a non-fluorinated polymer is infused into the pores of the ePTFE on at least a portion of the interior of the lumen at the open end of the lumen.

18. The ventricular catheter portion of claim 16, wherein a non-fluorinated liner is inserted into at least a portion of the interior of the open end of the lumen.

19. The ventricular catheter portion of claim 18, wherein the liner is comprised of polyurethane or silicone.

20. The ventricular catheter portion of any of claims 1 to 10, wherein at least a portion of a surface of the proximal end portion, or a portion of a surface of both the proximal end portion and the distal end portion is coated with one or more fluorinated liquids, one or more perfluorinated liquids, or a combination of one or more fluorinated liquids and/or one or more perfluorinated liquids.

21. The ventricular catheter portion of claim 20, wherein the fluorinated liquid, the perfluorinated liquid, or the combination of one or more fluorinated liquids and/or one or more perfluorinated liquids comprises a fluoroalkane or perfluoroalkane.

22. The ventricular catheter portion of claim 20, wherein greater than 50%, 75%, 80%, or 90%, or 100%, of an exterior surface of the proximal end portion, the distal end portion, or both the proximal end portion and the distal end portion is comprised of a fluoropolymer and/or perfluorinated polymer and is coated with the fluorinated liquid, perfluorinated liquid, or a combination of one or more fluorinated liquids and/or perfluorinated liquids.

23. The ventricular catheter portion of claim 20, wherein the one or more fluorinated liquids, the one or more perfluorinated liquids, or the combination of one or more fluorinated liquids and/or perfluorinated liquids comprises, consists essentially of, or consists of one or more perfluorinated liquids.

24. The ventricular catheter portion of claim 20, wherein the one or more fluorinated liquids, the one or more perfluorinated liquids, or the combination of one or more fluorinated liquids and/or one or more perfluorinated liquids comprises greater than:
(i) 50%, 60%, 70%, 80%, 90%, 95%, or 98% of one or two fluorinated and/or perfluorinated compounds on a weight basis;
(ii) 50%, 60%, 70%, 80%, 90%, 95%, or 98% of fluorinated compounds on a weight basis;
(iii) 50%, 60%, 70%, 80%, 90%, 95%, or 98% of perfluorinated compounds on a weight basis; or
(iv) 50%, 60%, 70%, 80%, 90%, 95%, or 98% of fluorinated and/or perfluorinated compounds on a weight basis.

25. The ventricular catheter portion of claim 20, wherein the one or more fluorinated liquids, the one or more perfluorinated liquids, or the combination of one or more fluorinated liquids and/or one or more perfluorinated liquids comprises: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexa)fluoropropylene oxide, or combinations thereof.

26. A method of making a ventricular catheter portion of a medical device according to any of claims 1 to 10, the method comprising preparing all or part of the proximal end portion or the distal end portion by one or more of: machining away excess material, drilling, laser cutting, laser drilling, extrusion, co-extrusion, injection molding, compression molding, melt spinning, electrospinning, dip coating, blowing, foaming, over-molding, and chemical vapor deposition.

27. The method of claim 26, wherein the proximal portion and/or distal end portion comprise a fluoropolymer and/or perfluoropolymer present on at least a portion of their exterior surfaces, wherein the fluoropolymer or perfluoropolymer containing exterior surface is formed by a method selected from the group consisting of: extrusion, co-extrusion, injection molding, compression molding, melt spinning, electrospinning, dip coating, blowing, and/or foaming.

28. A ventricular catheter portion of a hydrocephalus shunt made by the method of claim 26.

29. The ventricular catheter portion of a hydrocephalus shunt of any of claims 1 to 10 packaged in a sterile nonporous container coated with one or more fluorinated liquids, one or more perfluorinated liquids, or a combination of one or more fluorinated liquids and/or one or more perfluorinated liquids.

30. The ventricular catheter portion of a hydrocephalus shunt of any of claims 1 to 10, packaged in a sterile nonporous container in the absence of one or more fluorinated liquids, one or more perfluorinated liquids, or a combination of one or more fluorinated liquids and/or one or more perfluorinated liquids.

31. The ventricular catheter portion of a hydrocephalus shunt of claim 29, where the package either further comprises or is accompanied by a sterile aliquot of the one or more fluorinated liquids, one or more perfluorinated liquids, or the combination of one or more fluorinated liquids and/or one or more perfluorinated liquids.

32. A hydrocephalus shunt comprising a ventricular catheter portion of any of claims 1-10.

33. The hydrocephalus shunt of claim 32, wherein the ventricular catheter portion is attached to a valve at the open end of the lumen permitting the lumen of the ventricular catheter portion to be in fluid communication with a distal catheter.

34. The hydrocephalus shunt of claim 33, wherein a portion of the valve providing fluid communication with the lumen is a hollow tube inserted into the open end of the lumen.

35. The hydrocephalus shunt of claim 33, wherein the portion of the valve inserted into the open end of the lumen has a barbed exterior surface and the compressive mechanical force of the tubing of the distal end on the barbed exterior surface secures the valve to the tubing.

36. A method of treatment comprising implanting the hydrocephalus shunt of claim 32 in an animal.

37. The method of claim 36, wherein the ventricular catheter portion of the shunt is implanted in the ventricle of a mammalian brain and ventricle pressure is reduced due to fluid flow out of the ventricle through the catheter.
